(19) United States
(12) Patent Application Publication
(10) Pub. No.: US 2022/0112168 A1
(43) Pub. Date: Apr. 14, 2022

(54) Environment-friendly preparation method of a substituted oxazole compound

(71) Applicant: XIN FA PHARMACEUTICAL CO., LTD., Dongying (CN)

(72) Inventors: Xinfa LI, Dongying (CN); Lishan ZHOU, Dongying (CN); Chengwei WANG, Dongying (CN); Ningning LIU, Dongying (CN); Yuxin QI, Dongying (CN)

(21) Appl. No.: 17/643,391

(22) Filed: Dec. 8, 2021

Related U.S. Application Data
(63) Continuation of application No. PCT/CN2020/120528, filed on Oct. 13, 2020.

(30) Foreign Application Priority Data
Oct. 12, 2020 (CN) 202011086310.1

(51) Int. Cl. C07D 263/42

(52) U.S. Cl. CPC C07D 263/42

(57) ABSTRACT

N-substituted formyl-alpha-substituted glycine ester is used as the initial raw material, a cyclization reaction is performed under the action of a dehydrating agent (trisubstituted phosphine dihalide, a combination of trisubstituted phosphine dihalide and an acyl halide reagent, or a combination of trisubstituted phosphine oxide and an acyl halide reagent) and an organic amine to obtain the substituted oxazole compound, and the resulting substituted oxazole compound can be further saponified and de-carboxylated to obtain a medical intermediate 4-substituent-5-substituent oxy-oxazole; the reaction process can be carried out in a continuous flow mode to improve the productivity and reduce operations; the byproduct trisubstituted phosphine oxide in the reaction process can be repeatedly used to reduce the cost; dehydrating agents (phosphorus oxychloride and phosphorus pentoxide).

### CROSS REFERENCES

This application is the Continuation application of International Application No. PCT/CN2020/120528 filed on 13 Oct. 2020 which designated the U.S. and claims priority to Chinese Application Nos. 202011086310.1 filed on 12 Oct. 2020, the entire contents of each of which are hereby incorporated by reference.

### FIELD OF THE INVENTION

The present invention relates to an environment-friendly preparation method of a substituted oxazole compound and belongs to the field of medical biochemistry engineering.

### BACKGROUND OF THE INVENTION

Oxazole compounds are crucial intermediates for the preparation of bio-active substances. 4-methyl-5-alkoxy oxazole, for example, is an important intermediate in the synthesis of vitamin B6, one of the essential vitamins that play a key role in animal and human growth. Therefore, 4-methyl-5-alkoxy oxazole has wide applications in various fields, including pharmaceutical, food, feed additive, and cosmetic industry.

For now, the oxazole compound 4-methyl-5-alkoxy oxazole (taking 4-methyl-5-ethoxy oxazole as an example) are mainly prepared by the following two methods:

1. Cyclization of Formyl Compounds

In literatures J. Am. Chem. Soc. 2007, 129, 4440-4455 and European Journal of Medicinal Chemistry 62 (2013) 486-487, ethyl 2-aminopropanoate was formylated to ethyl N-formyl-2-aminopropanoate and cyclized under the action of phosphorus pentoxide or other dehydrating agents to obtain 4-methyl-5-ethoxy oxazole. This preparation method consumes a large amount of phosphorus pentoxide during cyclization. It produces much wastewater and a low yield. Also, its high costs and difficult operation hinder the industrial expansion.

2. Cyclization of Oxalyl Compounds

In Chinese patent literatures CN86101512A, CN102321043 A, and CN103435568 A and the literature “Chinese Journal of Pharmaceuticals 2009, 40 (2) 81-82, 96”, N-Ethoxyoxoacetyl-L-alanine ethyl ester was used as a raw material to prepare the corresponding oxazole compound by cyclization. The specific process was as follows: L-alanine, excessive oxalic acid, ethyl alcohol, and benzene were used as raw materials and refluxed to prepare N-Ethoxyoxoacetyl-L-alanine ethyl ester, then cyclized in the presence of phosphorus oxychloride and organic base to obtain the oxazole compound and followed by hydrolysis and decarboxylation to obtain the 4-methyl-5-ethoxy oxazole. This method needs a prolonged reaction time, as it uses phosphorus oxychloride and other dehydrating agents for cyclization reaction. It produces a lot of viscous substances during the reaction, which increases the difficulty in stratification. The wastewater produced by it not only has a high COD and dark color, but also contains many complex salts of sodium phosphate, disodium hydrogen phosphate, and sodium chloride which are difficult to treat and not environment-friendly. Also, its production costs are high.

Kotobuki Pharmaceutical has disclosed in the UK patent literature GB1195854 a method for preparing 5-Ethoxy-4-methyl-2-oxazolic acid ethyl ester with phosgene and N-ethoxy oxalyl-alanine ethyl ester. This method uses phosgene/triethylamine/trichloromethane as the cyclization system to obtain the Compound of Formula I, a key intermediate for the preparation of vitamin B6, through dehydration reaction. Its yield can reach 80.1%. The use of phosgene or triphosgene can reduce the production and emission of phosphorus salts, but it prolongs the reaction time, results in incomplete conversion of raw materials and a number of side reactions, and increases the consumption of triethylamine due to the reaction between the phosgene and the triethylamine. All these disadvantages hinder the industrial expansion of the method.

### SUMMARY OF THE INVENTION

To address the drawbacks in the prior art, the present invention provides an environment-friendly preparation method of a substituted oxazole compound. According to the method, N-substituted formyl-alpha-substituted glycine ester is used as the initial raw material to prepare the substituted oxazole compound with the easily accessible trisubstituted phosphine dihalide, a combination of trisubstituted phosphine dihalide and an acyl halide reagent, or a combination of trisubstituted phosphine oxide and an acyl halide reagent as the dehydrating agent; the byproduct trisubstituted phosphine oxide in the reaction process can be repeatedly used to reduce the cost; dehydrating agents (phosphorus oxychloride and phosphorus pentoxide), which are high in price and generate a large amount of wastewater, are not used; no high-temperature cyclization reaction is needed. Such a method has a short reaction time, produces no viscous substance in the reaction process, and is easy to operate. As it discharges no phosphorus-containing wastewater in the technological process (the wastewater contains only one kind of salt, namely sodium chloride), it is environment-friendly and low-cost. Additionally, it is high in atom economy, high in target product yield and purity, and suitable for industrial applications.

### DEFINITION OF TERMS

Compound of Formula I: a substituted oxazole compound, namely 4-R₂ substituent-5-R₁ substituent oxy-2-R₃ substituent oxazole;

Compound of Formula II: N—R₃ substituent formyl-α-R₂ substituent glycine ester;

Compound of Formula III: 4-R₂ substituent-5-R₁ substituent oxy oxazole-2-carboxylate;

Compound of Formula IV: 4-R₂ substituent-5-R₁ substituent oxy oxazole;

Compound of Formula VI: RₐR_{b}R_{c} tri-substituent phosphine dihalide.

The numbering of the compounds in the description is completely consistent with the numbering of their structural formulas, and they have the same references.

A technical solution of the present invention is provided below:

An environment-friendly preparation method of a substituted oxazole compound, comprising the steps as follows:

A Compound of Formula II is dissolved in solvent A and cyclized to obtain the substituted oxazole compound (I) in the presence of a dehydrating agent and an organic amine; the dehydrating agent is trisubstituted phosphine dihalide, a combination of trisubstituted phosphine dihalide and an acyl halide reagent, or a combination of trisubstituted phosphine oxide and an acyl halide reagent.

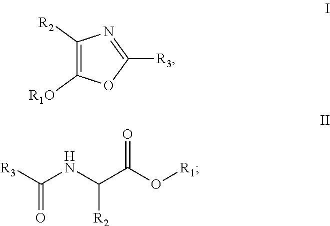

Structural formulas I and II of the compounds, wherein:

R₁ can be hydrogen, a CₙH₂ₙ₊₁ straight-chain or branched-chain group (1≤n≤10), aryl, or substituted aryl;

R₂ can be hydrogen, a CₙH₂ₙ₊₁ straight-chain or branched-chain group (1≤n≤10), aryl, or substituted aryl;

R₃ is —COOR, —CH₂COOR, or —CH₂CH₂COOR, where R is a CₙH₂ₙ₊₁ straight-chain or branched-chain group (1≤n≤10).

Preferably according to the present invention, in structural formulas I and II of the compounds, R₁ is methyl or ethyl, and R₂ is methyl.

Preferably according to the present invention, the said substituted oxazole compound is 4-R₂ substituent-5-R₁ substituent oxy-2-R₃ substituent oxazole; preferably, it is 4-methyl-5-alkoxy-2-R₃ substituent oxazole; more preferably, it is 4-methyl-5-ethyoxyl-2-R₃ substituent oxazole; still more preferably, it is 4-methyl-5-ethyoxyl-2-ethoxycarbonyl oxazole, 4-methyl-5-methoxyl-2-methoxycarbonyl oxazole, 4-phenyl-5-ethyoxyl-2-ethoxycarbonyl oxazole, or 5-ethyoxyl-2-ethoxycarbonyl oxazole.

Preferably according to the present invention, the said solvent A is one selected from among dichloromethane, chloroform, n-hexane, cyclohexane, petroleum ether, n-heptane, chlorobenzene, benzene, methylbenzene, dimethylbenzene, dimethylsulfoxide, trichloromethane, trichloroethane or dichloroethane, or a combination of two or more thereof; a mass ratio between the solvent A and the Compound of Formula II is (0.5-20.0):1; Preferably, the mass ratio between the solvent A and the Compound of Formula II is (3.0-10.0):1; more preferably, it is (4.0-9.0):1.

According to the present invention, when the said dehydrating agent is a combination of trisubstituted phosphine oxide and an acyl halide reagent, the trisubstituted phosphine oxide can react in situ with the acyl halide reagent to obtain the trisubstituted phosphine dihalide which will then play the role of dehydration.

According to the present invention, the structural formula of the said trisubstituted phosphine oxide is RₐR_{b}R_{c}P═O, where Rₐ, R_{b}, and R_{c} can be methyl, ethyl, as well as a C₃-C₁₀ straight-chain or branched-chain alkyl group, aryl, and substituted aryl and are preferred to be phenyl and isobutyl; Rₐ, R_{b}, and R_{c} can be either the same or different but are preferred to be the same.

Preferably, when Rₐ, R_{b}, and R_{c} are aryl groups, the trisubstituted phosphine oxide is of a structure as shown in the formula V below.

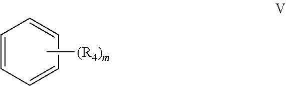

The structural formula as shown in formula V, wherein m is 0, 1, 2, 3, 4, or 5, and R₄ can be hydrogen, a CₙH₂ₙ₊₁ straight-bran or branched-chain alkyl group (1≤n≤10), or halogen; preferably, R₄ is hydrogen. The structural formula as shown in formula V represents that the trisubstituted phosphine oxide has m R₄ groups that are separately bonded to the six carbons of the benzene ring arbitrarily. The m substituents can be either the same or different but are preferred to be different.

Preferably, the said trisubstituted phosphine oxide is trialkyl phosphine oxide, triphenyl phosphine oxide, or tris(4-methylphenyl) phosphine oxide.

According to the present invention, the structural formula of the said trisubstituted phosphine dihalide is as shown in Formula VI:

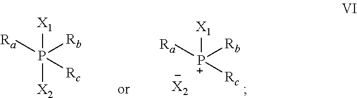

Wherein, Rₐ, R_{b}, and R_{c} can be methyl, ethyl, as well as C₃-C₁₀ straight-chain or branched-chain alkyl group, aryl, and substituted aryl and are preferred to be phenyl and isobutyl; Rₐ, R_{b}, and R_{c} can be either the same or different but are preferred to be different.

X₁ and X₂ can be fluorine, chlorine, bromine, or iodine and are preferred to be chlorine.

X₁ and X₂ can be either the same or different but are preferred to be different.

Preferably, when Rₐ, R_{b}, and R_{c} are aryl groups, the trisubstituted phosphine dihalide is of a structure as shown in the formula V below.

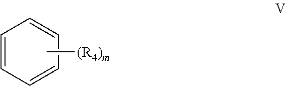

The structural formula as shown in formula V, wherein m is 0, 1, 2, 3, 4, or 5, and R₄ can be hydrogen, a CₙH₂ₙ₊₁ straight-bran or branched-chain alkyl group (1≤n≤10), or halogen; preferably, R₄ is hydrogen. The structural formula as shown in formula V represents that the trisubstituted phosphine dihalide has m R₄ groups that are separately bonded to the six carbons of the benzene ring arbitrarily. The m substituents can be either the same or different but are preferred to be different.

Preferably according to the present invention, the said trisubstituted phosphine dihalide is trialkyl phosphine dichloride, triphenyl phosphine dichloride, or tris(4-methylphenyl) phosphine dichloride.

Preferably according to the present invention, when the said dehydrating agent is trisubstituted phosphine dihalide, the molar ratio between the trisubstituted phosphine dihalide and the Compound of Formula II is (0.01-5.0):1; preferably, the molar ratio between the trisubstituted phosphine dihalide and the Compound of Formula II is (0.1-1.5):1; more preferably, it is (0.2-1.2):1.

When the said dehydrating agent is a combination of trisubstituted phosphine oxide and an acyl halide reagent, the molar ratio between the acyl halide reagent and the Compound of Formula II is (0.1-2.0):1, and that between the trisubstituted phosphine oxide and the Compound of Formula II is (0.01-5.0):1; preferably, the molar ratio between the said acyl halide reagent and the Compound of Formula II is (0.3-1):1; more preferably, it is (0.4-0.9):1; preferably, the molar ratio between the trisubstituted phosphine oxide and the Compound of Formula II is (0.1-1.5):1; more preferably, it is (0.2-1.3):1.

When the said dehydrating agent is a combination of trisubstituted phosphine dihalide and an acyl halide reagent, the molar ratio between the said acyl halide reagent and the Compound of Formula II is (0.1-2.0):1, and that between the trisubstituted phosphine dihalide and the Compound of Formula II is (0.01-5.0):1; Preferably, the molar ratio between the said acyl halide reagent and the Compound of Formula II is (0.3-1):1; more preferably, it is (0.5-0.8):1; preferably, the molar ratio between the trisubstituted phosphine dihalide and the Compound of Formula II is (0.1-1.5):1; more preferably, it is (0.3-1.4):1.

Preferably according to the present invention, the said acyl halide reagent is sulfuryl halide, thionyl halide, oxalyl halide, carbonyl halide, diphosgene, or triphosgene and is preferred to be carbonyl chloride or triphosgene.

Preferably according to the present invention, the said acyl halide reagent is an acyl chloride agent and is preferred to be sulfuryl chloride, thionyl chloride, oxalyl chloride, carbonyl chloride, phosgene, diphosgene, or triphosgene; more preferably, it is carbonyl chloride or triphosgene.

Preferably according to the present invention, when the combination of trisubstituted phosphine oxide and an acyl chloride reagent is used as the dehydrating agent, the substituted oxazole compound can be obtained with the batch synthesis process, and the said acyl halide reagent is dropwise added to the reaction system.

Preferably according to the present invention, when the combination of trisubstituted phosphine oxide and an acyl chloride reagent is used as the dehydrating agent, the substituted oxazole compound can be obtained with the continuous flow process by feeding the dehydrating agent/organic amine/compound II alone or a mixture of any two thereof continuously.

Preferably according to the present invention, when the trisubstituted phosphine dihalide compound is used as the dehydrating agent, the substituted oxazole compound can be obtained with the continuous flow process by feeding the dehydrating agent/organic amine/compound II alone or a mixture of any two thereof continuously.

Preferably according to the present invention, the continuous flow process used in the synthesis of the substituted oxazole compound can be carried out in a tank continuous reactor, a pipeline continuous reactor, a tower continuous reactor, a microchannel reactor, and so on.

Preferably according to the present invention, the said organic amine is trialkylamine, wherein the general formula of the alkyl is CₙH₂ₙ₊₁ (1≤n≤10); the alkyl is preferred to be methyl, ethyl, isopropyl, n-propyl, isobutyl, or n-butyl and more preferred to be ethyl, n-propyl, or n-butyl; the molar ratio between the said organic amine and the Compound of Formula II is (1.8-4.0):1; preferably, the molar ratio between the organic amine and the Compound of Formula II is (2.0-3.0):1; more preferably, it is (2.2-2.8):1.

Preferably according to the present invention, the Compound of Formula II is N-ethoxalyl glycine ethyl ester, N-ethoxalyl-α-alanine ethyl ester, N-ethoxalyl glycine methyl ester, N-butoxalyl-α-alanine butyl ester, N-ethoxalyl-α-alanine butyl ester, N-methoxalyl-α-alanine methyl ester, N-ethoxalyl-α-phenylglycine ethyl ester, or N-ethoxalyl-α-alanine methyl ester, or a combination of any two or more thereof.

Preferably according to the present invention, the cyclization reaction temperature is −20° C.-150° C.; preferably, the reaction temperature is 30-95° C.; more preferably, it is 35-70° C.;

Preferably, the cyclization reaction time is 0.2-10 hours; more preferably, it is 0.6-9 hours.

Preferably according to the present invention, the Compound of Formula II is cyclized to obtain a reaction liquid; the follow-up treatment of the reaction liquid comprises steps as follows: water is added to the reaction liquid for stratification; an aqueous phase and an organic phase are obtained by extracting the aqueous layer with solvent A and combining organic phases; the resulting organic phase is distilled under atmospheric pressure to recover the solvent A and then under a reduced pressure to obtain the substituted oxazole compound (I); the resulting aqueous phase or the residue of the reduced pressure distillation contains trisubstituted phosphine oxide; the trisubstituted phosphine oxide can be used together with an acyl halide reagent to prepare trisubstituted phosphine dihalide, which functions as a dehydrating agent, or recycled and used as a dehydrating agent directly; the aqueous phase can be neutralized by sodium hydroxide and distilled to recover organic amine.

According to the present invention, the organic amine is used as an acid-binding agent to react with a byproduct of the reaction process hydrogen chloride and prepare organic amine hydrochloride and then neutralized by sodium hydroxide to recover organic amine and the byproduct sodium chloride.

According to the present invention, the resulting substituted oxazole compound (I) can be further used to prepare the oxazole pharmaceutical intermediate Compound of Formula IV below according to the prior art.

According to the present invention, a preparation method of 4-substituted alkyl-5-substituent oxy oxazole is also provided; the said 4-substituted alkyl-5-substituent oxy oxazole is of a structure as shown in formula IV:

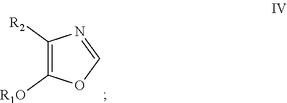

The method comprises steps as follows:

A Compound of Formula II is dissolved in solvent A and cyclized to obtain the substituted oxazole compound (I) in the presence of a dehydrating agent and an organic amine; the substituted oxazole compound (I) is then saponified and de-carboxylated to obtain 4-substituted alkyl-5-substituent oxy oxazole (IV);

The dehydrating agent is trisubstituted phosphine dihalide, a combination of trisubstituted phosphine dihalide and an acyl halide reagent, or a combination of trisubstituted phosphine oxide and an acyl halide reagent.

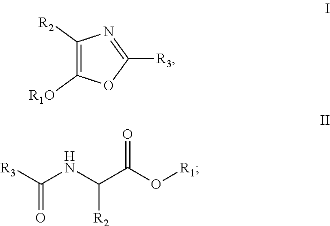

Structural formulas I and II of the compounds, wherein:

R₁ can be hydrogen, a CₙH₂ₙ₊₁ straight-chain or branched-chain group (1≤n≤10), aryl, or substituted aryl;

R₂ can be hydrogen, a CₙH₂ₙ₊₁ straight-chain or branched-chain group (1≤n≤10), aryl, or substituted aryl;

R₃ is —COOR, —CH₂COOR, or —CH₂CH₂COOR, wherein R is a CₙH₂ₙ₊₁ straight-chain or branched-chain group (1≤n≤10).

Preferably according to the present invention, the substituted oxazole compound (I) can be saponified in the presence of alkali to obtain the Compound of Formula III and then decarboxylated under acidic conditions to obtain the Compound of Formula IV.

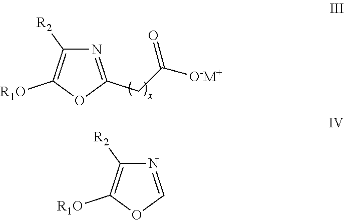

In the structural formula of the Compound of Formula III, the substituents R₁ and R₂ are the same as those in the structural formula of the Compound of Formula II; M is an alkali metal; x is 0, 1, or 2, wherein x=0 means that the COOM is directly bonded to the oxazole ring. In the structural formula of the Compound of Formula IV, the substituents R₁ and R₂ are the same as those in the structural formula of the Compound of Formula II.

According to the present invention, the said saponification reaction and decarboxylation reaction can be carried out in a way familiar to those skilled in the field.

Preferably, the said alkali is the aqueous solution of an alkali metal hydroxide with a mass concentration of 20-30%; the said alkali metal is preferred to be sodium or potassium; the molar ratio between the said alkali and the substituted oxazole compound (I) is 1-1.5:1; the temperature of the said saponification reaction is 20-30° C. The saponification reaction time is 10-60 minutes.

Preferably, the said acidic conditions are created by adjusting the reaction system to a pH of 1-2 through the use of aqueous acid (mass concentration 20-35%); the temperature of the said decarboxylation reaction is 50-70° C.; the time of the decarboxylation reaction is 10-60 minutes.

According to the present invention, a preferred embodiment for the preparation of the Compound of Formula IV, comprising steps as follows: the Compound of Formula II is cyclized to obtain a reaction liquid; then, water is added to the reaction liquid for stratification; the resulting aqueous layer is extracted with solvent A, and the Compound of Formula I is obtained after combining organic phases and recovering the solvent; then alkali is added to the residue for saponification reaction, and stratification is conducted at the end of the reaction; the resulting organic layer is washed with water; the aqueous layers are combined to obtain the resulting solution that contains the Compound of Formula III and decarboxylated by aqueous acid to obtain the Compound of Formula IV. After the Compound of Formula IV is separated, the remaining aqueous phase or organic phase contains trisubstituted phosphine oxide which can be used together with an acyl chloride reagent to prepare trisubstituted phosphine dihalide, which functions as a dehydrating agent, or recycled and used as a dehydrating agent directly.

The reaction path of the preparation method for a substituted oxazole compound in the present invention is provided as follows:

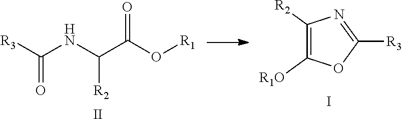

Where R₁ in the structural formula of the Compound of Formula II is hydrogen, a CₙH₂ₙ₊₁ group (1≤n≤10), aryl, or substituted aryl; R₂ is hydrogen, a CₙH₂ₙ₊₁ group (1≤n≤10), aryl, or substituted aryl; R₃ is —COOR, —CH₂COOR, or —CH₂CH₂COOR, wherein R is alkyl. When R₃ is —COOR, the Compound of Formula IV can be prepared through subsequent reactions. R₁, R₂, and R₃ in the structural formula of the Compound of Formula I, as well as R₁ and R₂ in the structural formula of the Compound of Formula IV are all consistent with those in the Compound of Formula II.

The present invention provides the following beneficial effects:
1. The present invention provides a novel and environment-friendly preparation method for a substituted oxazole compound via the cyclization reaction.

According to the method, N-substituted formyl (oxalyl)-alpha-substituted glycine ester (II) is used as the initial raw material, a cyclization reaction is performed under the action of a dehydrating agent (trisubstituted phosphine dihalide, a combination of trisubstituted phosphine dihalide and an acyl halide reagent, or a combination of trisubstituted phosphine oxide and an acyl halide reagent) and an organic amine to obtain the substituted oxazole compound (I), and the resulting substituted oxazole compound (I) can be further saponified and de-carboxylated according to the prior arts to obtain 4-substituted alkyl-5-substituent oxy oxazole (IV).
2. The whole reaction process of the present invention can be understood as follows: the Compound of Formula II is cyclized to remove one water molecule; the water molecule then reacts with trisubstituted phosphine dihalide to obtain trisubstituted phosphine oxide and two hydrogen halide molecules; the two hydrogen halide molecules react with the acid-binding agent organic amine to obtain the organic amine hydrochloride. The dehydrating agent in the present invention is trisubstituted phosphine dihalide, a combination of trisubstituted phosphine dihalide and an acyl halide reagent, or a combination of trisubstituted phosphine oxide and an acyl halide reagent. When the dehydrating agent is a combination of trisubstituted phosphine dihalide and an acyl halide reagent, the trisubstituted phosphine dihalide is converted into trisubstituted phosphine oxide after dehydration, and the trisubstituted phosphine oxide reacts with the acyl halide reagent in situ to obtain trisubstituted phosphine dihalide to continue with the dehydration reaction. When the dehydrating agent is a combination of trisubstituted phosphine oxide and an acyl halide reagent, the acyl halide reagent reacts with the trisubstituted phosphine oxide in situ to obtain trisubstituted phosphine dichloride, followed by the cyclization reaction; the reaction process produces only sodium chloride and byproduct gases, such as sulfur dioxide or carbon dioxide, and as it produces less wastewater and waste gas, it is very environment-friendly. The dehydrating agents in the present invention are easily accessible; the preparation of the substituted oxazole compound produces trisubstituted phosphine oxide as a byproduct to achieve repeated use, which facilitates the quantitative conversion to trisubstituted phosphine dichloride, reduces the cost by recycling materials, and agrees with the concept of environmental protection and atom economy. Dehydrating agents (phosphorus oxychloride and phosphorus pentoxide), which are high in price and generate a large amount of wastewater, are not used in the present invention, and no high-temperature cyclization reaction is needed. Such a method has a simple process and is easy to operate. As it discharges no phosphorus-containing wastewater in the technological process, it is also environment-friendly and low-cost.
3. The method in the present invention has high reactivity, good reaction selectivity, high atom economy, and high yield and purity. Its yield is more than 95% and purity is more than 99%, and it is suitable for industrial application. The resulting substituted oxazole compound (I) can be further saponified and de-carboxylated according to the prior arts to obtain the oxazole pharmaceutical intermediate (IV).

### EMBODIMENTS

Hereinafter, the present invention will be illustrated in detail with reference to the embodiments; however, the present invention is not limited thereto.

The percentages in the embodiments all refer to mass percentages, unless otherwise indicated.

The yields in the embodiments all refer to molar yields.

The raw materials and reagents in the embodiments are all commercially available. The raw material N-ethoxalyl-α-alanine ethyl ester is supplied by Xinfa Pharmaceutical Co., Ltd.

Gas-phase detection in the invention uses a Shimadzu gas chromatograph (model GC-1020 PLUS) for reaction monitoring and purity testing. Part of the purity is tested by high performance liquid chromatography (marked as HPLC).

### 1. Embodiment 1: Preparation of 4-methyl-5-ethyoxyl-2-ethoxycarbonyl oxazole (I₁)

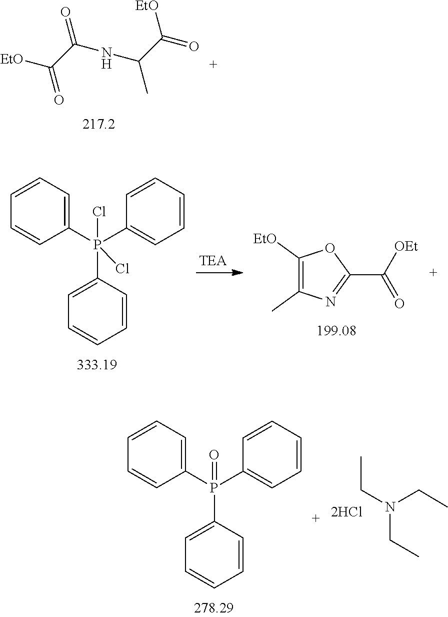

In the mixing tank, 6000 g of well-prepared methylbenzene solution of triphenyl phosphine dichloride (containing 1000 g of triphenyl phosphine dichloride) and 650 g of N-ethoxalyl-α-alanine ethyl ester are added and evenly mixed; then, the reaction mixture is fed into the flow reactor at a rate of 111 g/min to react under a temperature of 35-75° C. with the triethylamine pumped in at a rate of 11 g/min; the solid-liquid mixture flowing out of the reactor is hydrolyzed and stratified by 2000 ml of water; the resulting aqueous phase is extracted by 2000 ml of methylbenzene; the methylbenzene phases are combined and distilled at reduced pressure; after the solvent is recovered, 578.9 g of 4-methyl-5-ethyoxyl-2-ethoxycarbonyl oxazole is obtained through high vacuum distillation in a yield of 96% and a GC purity of 99%.

The main composition of the residue after the reduced pressure distillation is triphenyl phosphine oxide, which can be used repeatedly to prepare the dehydrating agent.

Nuclear magnetic resonance data of the resulting product are provided below:

¹H NMR (CDCl₃, δ, ppm):

4.28 (q, 2H), 4.31 (q, 2H), 2.07 (s, 3H), 1.36 (t, 3H), 1.33 (t, 3H).

4-methyl-5-ethyoxyl oxazole can be obtained by further reaction following a usual method:

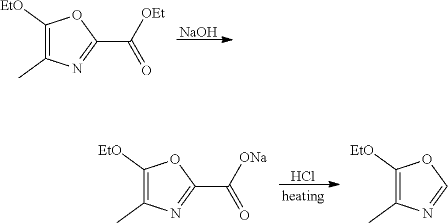

In a reaction flask, 201 g of the resulting 4-methyl-5-ethyoxyl-2-ethoxycarbonyl oxazole and 270 g of 15% liquid caustic soda are added; then the reaction mixture is distilled at a reduced pressure to recover ethyl alcohol; then 15% hydrochloric acid is dropwise added to adjust the pH value to 2.5, and the reaction system is heated up to 60-62° C. until no gas escapes; then liquid caustic soda is added to the reaction system, and steam distillation and stratification are conducted; after the resulting oil layer is dried by anhydrous sodium sulfate, 117 g of 4-methyl-5-ethyoxyl oxazole is obtained.

### Embodiment 2: Preparation of 4-methyl-5-ethyoxyl-2-ethoxycarbonyl oxazole (I₁)

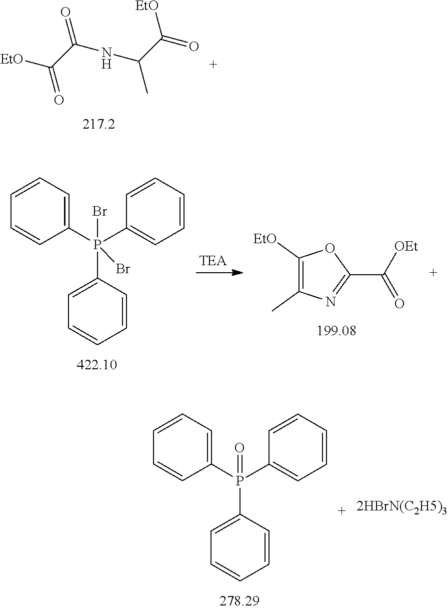 [SMILES: CCOC(=O)C(=O)NC(C)C(=O)OCC ; BrP(Br)(c1ccccc1)(c1ccccc1)c1ccccc1 ; CCOC(=O)c1nc(C)c(OCC)o1 ; O=P(c1ccccc1)(c1ccccc1)c1ccccc1]

In the mixing tank, 6000 g of well-prepared methylbenzene solution of triphenyl phosphine dibromide (containing 1300 g of triphenyl phosphine dibromide) and 650 g of N-ethoxalyl-α-alanine ethyl ester are added and evenly mixed; then, the reaction mixture is fed into the flow reactor at a rate of 111 g/min to react under a temperature of 35-75° C. with the triethylamine pumped in at a rate of 11 g/min; the solid-liquid mixture flowing out of the reactor is hydrolyzed and stratified by 2000 ml of water; the resulting aqueous phase is extracted by 2000 ml of methylbenzene; the methylbenzene phases are combined and distilled at reduced pressure; after the solvent is recovered, 572 g of 4-methyl-5-ethyoxyl-2-ethoxycarbonyl oxazole is obtained through high vacuum distillation in a yield of 94.8% and a GC purity of 99%.

The main composition of the residue after the reduced pressure distillation is triphenyl phosphine oxide, which can be used repeatedly to prepare the dehydrating agent.

### 1. Comparative Example 1: Preparation of 4-methyl-5-ethyoxyl-2-ethoxycarbonyl oxazole (I₁)

In a 250 ml flask, 100 g of trichloromethane, 10 g of solid phosgene, and 21.7 g (0.1 mol) of N-ethoxalyl-α-alanine ethyl ester are added; then, 25 g of triethylamine is dropwise added within 2 hours under 0-10° C. to have the reaction system react for 1 h under 0-10° C., and 30 g of water is added for stratification; the resulting aqueous layer is extracted twice by trichloromethane (a total consumption of 30 g); the organic phases are combined and distilled under the atmospheric pressure to recover trichloromethane; then after a reduced pressure distillation, 14.5 g of 4-methyl-5-ethyoxyl-2-ethoxycarbonyl oxazole is obtained in a yield of 71.6% and a GC purity of 98.3%.

It can be seen from the comparative example that the product yield is low when phosgene is used as the dehydrating agent.

### Embodiment 3: Preparation of 4-methyl-5-ethyoxyl-2-ethoxycarbonyl oxazole (I₁)

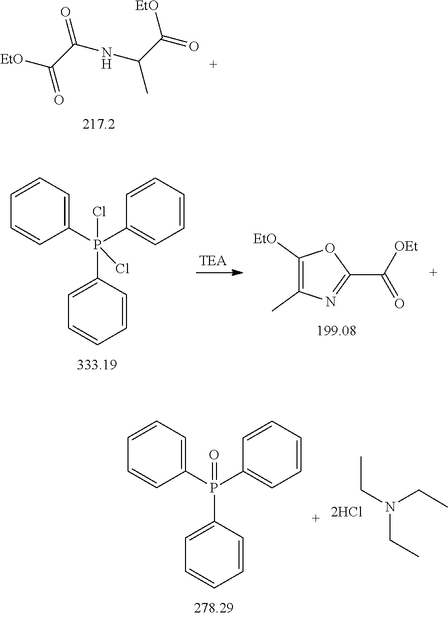

In a 250 ml flask, 100 g of trichloromethane, 33.3 g (0.1 mol) of triphenyl phosphine dichloride, and 21.7 g (0.1 mol) N-ethoxalyl-α-alanine ethyl ester are added; then, 20.2 g (0.2 mol) of triethylamine is dropwise added within 2 hours under 20-25° C. to have the reaction system react for 1 h under 35-40° C., and, after the reaction of raw materials ends, 30 g of water is added for stratification; the resulting aqueous layer is extracted twice by trichloromethane (a total consumption of 30 g); the organic phases are combined and distilled under the atmospheric pressure to recover trichloromethane; then after a reduced pressure distillation, 18.8 g of 4-methyl-5-ethyoxyl-2-ethoxycarbonyl oxazole is obtained in a yield of 94.4% and a GC purity of 99.9%. The main composition of the residue after the reduced pressure distillation is triphenyl phosphine oxide, which can be used repeatedly as the dehydrating agent.

Nuclear magnetic resonance data of the resulting product are provided below:

¹H NMR (CDCl₃, δ, ppm):

4.28 (q, 2H), 4.31 (q, 2H), 2.07 (s, 3H), 1.36 (t, 3H), 1.33 (t, 3H).

### Embodiment 4: Preparation of 4-methyl-5-ethyoxyl-2-ethoxycarbonyl oxazole

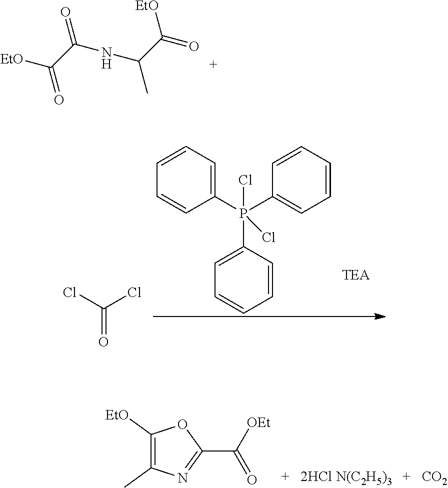

In a 500 ml 4-neck flask, 100 g of methylbenzene, 3.4 g (0.01 mol) of triphenyl phosphine dichloride, 21.7 g (0.1 mol) of N-ethoxalyl-α-alanine ethyl ester, and 20.8 g (0.206 mol) of triethylamine are added; then, the solution of 9.9 g (0.1 mol) of phosgene and 50 g of methylbenzene is dropwise added within 2 hours under 25-30° C. to have the reaction system react for 1 h under 65-70° C., and, after the reaction of raw materials ends, 30 g of water is added for stratification; the resulting aqueous layer is extracted twice by methylbenzene (a total consumption of 30 g); the organic phases are combined and distilled under the atmospheric pressure to recover methylbenzene; then after a reduced pressure distillation, 18.9 g of 4-methyl-5-ethyoxyl-2-ethoxycarbonyl oxazole is obtained in a yield of 94.9% and a GC purity of 99.9%. The main composition of the residue after the reduced pressure distillation is triphenyl phosphine oxide, which can be used repeatedly as the dehydrating agent.

### Embodiment 5: Preparation of 4-methyl-5-ethyoxyl-2-ethoxycarbonyl oxazole (I₁)

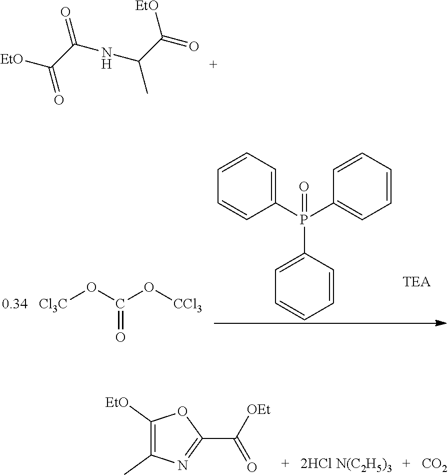

In a 500 ml 4-neck flask, 100 g of methylbenzene, 27.8 g (0.1 mol) of triphenyl phosphine oxide, and 21.7 g (0.1 mol) of N-ethoxalyl-α-alanine ethyl ester are added; then, the solution of 100 g of methylbenzene and 10.0 g (0.034 mol) of triphosgene and 24.3 g (0.24 mol) of triethylamine are dropwise added within 2 hours under 20-25° C. to have the reaction system react for 1 h under 45-50° C., and, after the reaction of raw materials ends, 30 g of water is added for stratification; the resulting aqueous layer is extracted twice by methylbenzene (a total consumption of 30 g); the organic phases are combined and distilled under the atmospheric pressure to recover methylbenzene; then after a reduced pressure distillation, 19.1 g of 4-methyl-5-ethyoxyl-2-ethoxycarbonyl oxazole is obtained in a yield of 96% and a GC purity of 99.9%. The main composition of the residue after the reduced pressure distillation is triphenyl phosphine oxide, which can be used repeatedly as the dehydrating agent.

### Embodiment 6: Preparation of 4-methyl-5-ethyoxyl-2-ethoxycarbonyl oxazole (I₁) (Through the Use of the Recovered Triphenyl Phosphine Oxide)

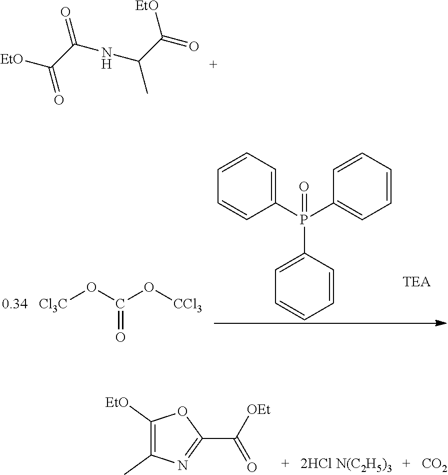

As described in Embodiment 5, the main composition of the resulting residue after 4-methyl-5-ethyoxyl-2-ethoxycarbonyl oxazole is distilled and recovered is triphenyl phosphine oxide, which can be used repeatedly as the dehydrating agent. In a 500 ml 4-neck flask, 100 g of methylbenzene is dissolved and added and then 21.7 g (0.1 mol) of N-ethoxalyl-α-alanine ethyl ester and 24.4 g (0.24 mol) of triethylamine are added; then, the solution of 100 g of methylbenzene and 9.9 g (0.033 mol) of triphosgene is dropwise added within 2 hours under 25-30° C. to have the reaction system react for 1 h under 45-50° C., and, after the reaction of raw materials ends, 30 g of water is added for stratification; the resulting aqueous layer is extracted twice by methylbenzene (a total consumption of 30 g); the organic phases are combined and distilled under the atmospheric pressure to recover methylbenzene; then after a reduced pressure distillation, 19.3 g of 4-methyl-5-ethyoxyl-2-ethoxycarbonyl oxazole is obtained in a yield of 96.9% and a GC purity of 99.9%. The main composition of the residue after the reduced pressure distillation is triphenyl phosphine oxide, which can be used repeatedly as the dehydrating agent.

### 1. Embodiment 7: Preparation of 4-methyl-5-methoxyl-2-methoxycarbonyloxazole (I₂) and 4-methyl-5-methoxy oxazole (IV₂)

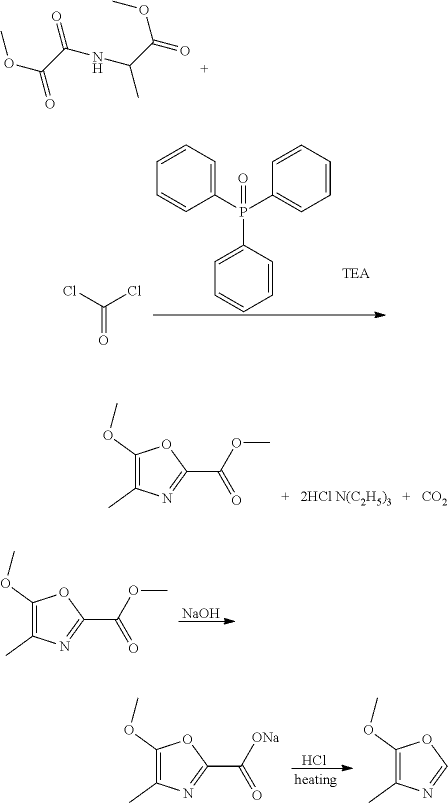

In a 500 ml 4-neck flask, 80 g of dimethylbenzene, 27.8 g (0.1 mol) of triphenyl phosphine oxide, 18.9 g (0.1 mol) of N-methoxalyl-α-alanine methyl ester, and 20.8 g (0.206 mol) of triethylamine are added; then, the mixed solution of 60 g of dimethylbenzene and 9.9 g (0.1 mol) of phosgene is dropwise added within 2 hours under 30-35° C. to have the reaction system react for 1 h under 35-40° C., and, after the reaction of raw materials ends, 30 g of water is added for stratification; the resulting aqueous layer is extracted twice by dimethylbenzene (a total consumption of 30 g); the organic phases are combined; upon GC testing, 16.1 g of 4-methyl-5-methoxyl-2-methoxycarbonyl oxazole is obtained in a yield of 94%.

To the combined organic phase, 20 g of 25% aqueous solution of sodium hydroxide is added, and then the reaction mixture is stirred and stratified under room temperature for 30 minutes; the resulting organic phase is washed twice by water (30 g each) to obtain triphenyl phosphine oxide, which can be repeatedly used as the dehydrating agent; then the aqueous layers are combined, and 24.7 g of 31% hydrochloric acid is added to adjust the pH to 1.5; the aqueous solution is then heated up to 60° C. and maintained at the temperature for 30 minutes before being neutralized to neutral; after a reduced pressure distillation, 10.1 g of 4-methyl-5-methoxy oxazole is obtained in a yield of 89.3 and a GC purity of 99.9% if calculated by N-methoxalyl-α-alanine methyl ester.

Nuclear magnetic resonance data of the resulting product 4-methyl-5-methoxy oxazole are provided below:

¹H NMR (CDCl₃, δ, ppm):

7.33 (s, 1H), 3.89 (s, 3H), 1.99 (s, 3H).

### Embodiment 8: Preparation of 4-methyl-5-methoxyl-2-methoxycarbonyloxazole (I₂) (Through the Use of the Recovered Triphenyl Phosphine Oxide)

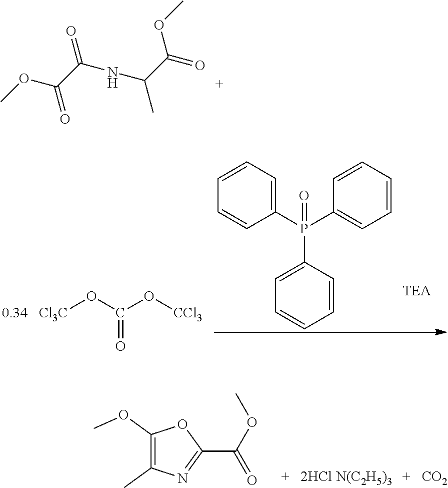

As described in Embodiment 5, the main composition of the resulting residue after 4-methyl-5-ethyoxyl-2-ethoxycarbonyl oxazole is distilled and recovered is triphenyl phosphine oxide, which can be used repeatedly as the dehydrating agent. In a 500 ml 4-neck flask, 100 g of methylbenzene is dissolved and added and then 18.9 g (0.1 mol) of N-methoxalyl-α-alanine methyl ester and 20.8 g (0.206 mol) of triethylamine are added; then, the solution of 60 g of methylbenzene and 9.9 g (0.033 mol) of triphosgene is dropwise added within 2 hours under 20-25° C. to have the reaction system react for 1 h under 35-40° C., and, after the reaction of raw materials ends, 30 g of water is added for stratification; the resulting aqueous layer is extracted twice by methylbenzene (a total consumption of 30 g); the organic phases are combined and distilled under the atmospheric pressure to recover methylbenzene; then after a reduced pressure distillation, 16.5 g of 4-methyl-5-methoxyl-2-methoxycarbonyl oxazole is obtained in a yield of 96.4%.

Nuclear magnetic resonance data of the resulting product are provided below:

¹H NMR (CDCl₃, δ, ppm):

3.89 (s, 3H), 3.85 (s, 3H), 2.07 (s, 3H)

### Embodiment 9: Preparation of 4-methyl-5-methoxyl-2-methoxycarbonyl oxazole (I₂)

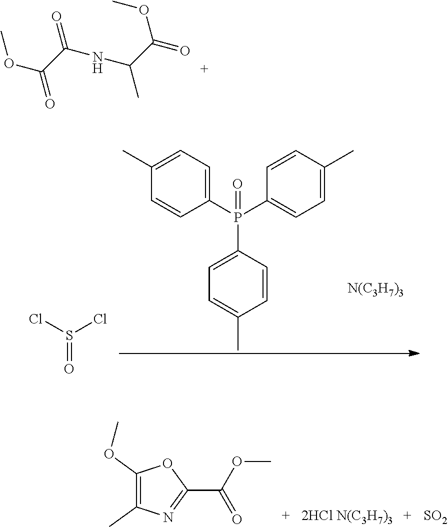

In a 500 ml 4-neck flask, 80 g of cyclohexane, 32 g (0.1 mol) of tris(4-methylphenyl)phosphine oxide, 18.9 g (0.1 mol) of N-methoxalyl-α-alanine methyl ester, and 28.9 g (0.202 mol) of tri-n-propylamine are added; then, the mixed solution of 60 g of cyclohexane and 11.9 g (0.1 mol) of thionyl chloride is dropwise added within 2 hours under 20-25° C. to have the reaction system react for 1 h under 60-65° C., and, after the reaction of raw materials ends, 30 g of water is added for stratification; the resulting aqueous layer is extracted twice by cyclohexane (a total consumption of 30 g); the organic phases are combined, washed once with 30 g of water, and stratified to obtain aqueous phase and organic phase; the aqueous phases are combined (this part of water can be used as the aqueous phases contain tris(4-methylphenyl)phosphine oxide); the resulting organic phase is distilled under the atmospheric pressure to recover cyclohexane; after a reduced pressure distillation, 15.6 g of 4-methyl-5-methoxyl-2-methoxycarbonyl oxazole is obtained in a yield of 91.2% and a GC purity of 99.2%.

### 1. Embodiment 10: Preparation of 4-phenyl-5-ethyoxyl-2-ethoxycarbonyl oxazole (I₃)

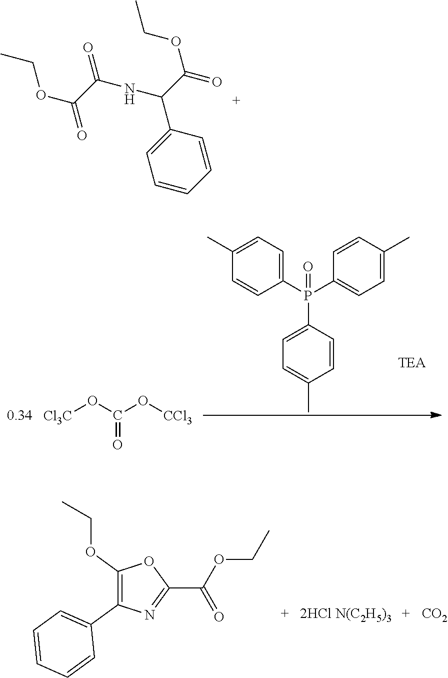

In a 500 ml 4-neck flask, 100 g of methylbenzene, 32 g (0.1 mol) of tris(4-methylphenyl)phosphine oxide, 27.9 g (0.1 mol) of N-ethoxalyl-α-phenylglycine ethyl ester, and 25.3 g (0.25 mol) of triethylamine are added; then, the solution of 50 g of methylbenzene and 10.1 g (0.034 mol) of triphosgene is dropwise added within 2 hours under 25-30° C. to have the reaction system react for 1 h under 45-50° C., and, after the reaction of raw materials ends, 30 g of water is added for stratification; the resulting aqueous layer is extracted by methylbenzene (a total consumption of 30 g); the organic phases are combined, washed once with 30 g of water, and stratified to obtain aqueous phase and organic phase; the aqueous phases are combined; the resulting organic phase is distilled under the atmospheric pressure to recover methylbenzene; then after a reduced pressure distillation, 22.3 g of 4-phenyl-5-ethyoxyl-2-ethoxycarbonyl oxazole is obtained in a yield of 85.3%.

Nuclear magnetic resonance data of the resulting product are provided below:

¹H NMR (CDCl₃, δ, ppm):

7.6 (d, 2H), 7.4-7.5 (m, 3H), 4.27 (q, 2H), 4.31 (q, 2H), 1.35 (t, 3H), 1.33 (t, 3H)

### Embodiment 11: Preparation of 5-ethyoxyl-2-ethoxycarbonyl oxazole (I₄)

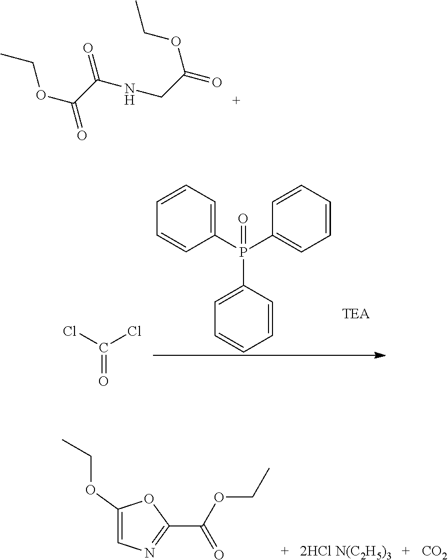

In a 500 ml 4-neck flask, 80 g of methylbenzene, 27.8 g (0.1 mol) of triphenyl phosphine oxide, 20.3 g (0.1 mol) of N-ethoxalyl glycine ethyl ester, and 22.2 g (0.22 mol) of triethylamine are added; then, the solution of 50 g of methylbenzene and 9.9 g (0.1 mol) of phosgene is dropwise added within 2 hours under 25-30° C. to have the reaction system react for 1 h under 50-55° C., and, after the reaction of raw materials ends, 30 g of water is added for stratification; the resulting aqueous layer is extracted by methylbenzene (a total consumption of 30 g); the organic phases are combined, washed once with 30 g of water, and stratified to obtain aqueous phase and organic phase; the aqueous phases are combined; the resulting organic phase is distilled under reduced pressure to recover methylbenzene; then after a reduced pressure distillation, 17.6 g of 5-ethyoxyl-2-ethoxycarbonyl oxazole is obtained in a yield of 95.1% and a GC purity of 99.9%.

Nuclear magnetic resonance data of the resulting product are provided below:

¹H NMR (CDCl₃, δ, ppm):

6.81 (s, 1H), 4.29 (q, 2H), 4.32 (q, 2H), 1.35 (t, 3H), 1.32 (t, 3H).

### 1. Embodiment 12: Preparation of 4-methyl-5-ethyoxyl-2-ethoxycarbonyl oxazole (I₁)

In a 250 ml flask, 100 g of trichloromethane, 33.3 g (0.1 mol) of triphenyl phosphine dichloride, and 21.7 g (0.1 mol) of N-ethoxalyl-α-alanine ethyl ester are added; then, 20.2 g (0.2 mol) of triethylamine is dropwise added within 2 hours under −40° C.-−45° C. to have the reaction system react for 1 h under −40° C.-−45° C.; then 30 g of water is added to quench the reaction for stratification under room temperature; the resulting aqueous layer is extracted twice by trichloromethane (a total consumption of 30 g); the organic phases are combined and distilled under the atmospheric pressure to recover trichloromethane; then after a reduced pressure distillation, 15.3 g of 4-methyl-5-ethyoxyl-2-ethoxycarbonyl oxazole is obtained in a yield of 76.6% and a GC purity of 98.3%.

As can be seen from the embodiment, the conversion and yield of the product are reduced when the temperature is low.

What is claimed is:

1. A preparation method of a substituted oxazole compound, comprising the steps as follows:
a Compound of Formula II is dissolved in solvent A and cyclized to obtain the substituted oxazole compound (I) in the presence of a dehydrating agent and an organic amine; the dehydrating agent is trisubstituted phosphine dihalide, a combination of trisubstituted phosphine dihalide and an acyl halide reagent, or a combination of trisubstituted phosphine oxide and an acyl halide reagent; 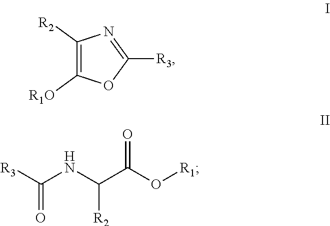
Structural formulas I and II of the compounds, wherein:
R₁ can be hydrogen, a CₙH₂ₙ₊₁ straight-chain or branched-chain group (1≤n≤10), aryl, or substituted aryl;
R₂ can be hydrogen, a CₙH₂ₙ₊₁ straight-chain or branched-chain group (1≤n≤10), aryl, or substituted aryl;
R₃ is —COOR, —CH₂COOR, or —CH₂CH₂COOR, wherein R is a CₙH₂ₙ₊₁ straight-chain or branched-chain group (1≤n≤10).

2. The preparation method of a substituted oxazole compound according to claim 1, characterized in that, in structural formulas I and II of the compounds, R₁ is methyl or ethyl, and R₂ is methyl.

3. The preparation method of a substituted oxazole compound according to claim 1, characterized in that the said solvent A is one selected from among dichloromethane, chloroform, n-hexane, cyclohexane, petroleum ether, n-heptane, dimethylbenzene, chlorobenzene, benzene, methylbenzene, dimethylsulfoxide, trichloromethane, trichloroethane or dichloroethane, or a combination of two or more thereof;
preferably, the mass ratio between the solvent A and the Compound of Formula II is (0.5-20.0):1.

4. The preparation method of a substituted oxazole compound according to claim 1, characterized in that the said organic amine is trialkylamine, wherein the general formula of the alkyl is CₙH₂ₙ₊₁ (1≤n≤10);
the alkyl is preferred to be methyl, ethyl, isopropyl, n-propyl, isobutyl, or n-butyl and more preferred to be ethyl, n-propyl, or n-butyl;
the organic amine is more preferred to be triethylamine;
preferably, the molar ratio between the said organic amine and the Compound of Formula II is (1.8-4.0):1; more preferably, it is (2.0-3.0):1.

5. The preparation method of a substituted oxazole compound according to claim 1, characterized in that the structural formula of the said trisubstituted phosphine oxide is RₐR_{b}R_{c}P═O;
wherein, Rₐ, R_{b}, and R_{c} can be methyl, ethyl, as well as a C₃-C₁₀ straight-chain or branched-chain alkyl group, aryl, and substituted aryl and are preferred to be phenyl and isobutyl; Rₐ, R_{b}, and R_{c} can be either the same or different;
preferably, when Rₐ, R_{b}, and R_{c} are aryl groups, the trisubstituted phosphine oxide is of a structure as shown in the formula V below: 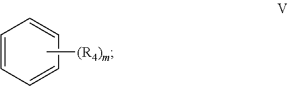
the structural formula as shown in formula V, wherein m is 0, 1, 2, 3, 4, or 5, R₄ can be hydrogen, a CₙH₂ₙ₊₁ straight-bran or branched-chain alkyl group (1≤n≤10), or halogen;
preferably, R₄ is hydrogen.

6. The preparation method of a substituted oxazole compound according to claim 5, characterized in that the said trisubstituted phosphine oxide is trialkyl phosphine oxide, triphenyl phosphine oxide, or tris(4-methylphenyl) phosphine oxide.

7. The preparation method of a substituted oxazole compound according to claim 1, characterized in that the structural formula of the said trisubstituted phosphine dihalide is as shown in Formula VI: 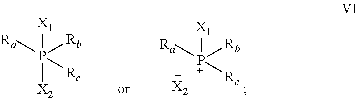
wherein, Rₐ, R_{b}, and R_{c} can be methyl, ethyl, as well as C₃-C₁₀ straight-chain or branched-chain alkyl group, aryl, and substituted aryl and are preferred to be phenyl and isobutyl; Rₐ, R_{b}, and R_{c} can be either the same or different;
X₁ and X₂ can be fluorine, chlorine, bromine, or iodine, and they can be either the same or different;
preferably, when Rₐ, R_{b}, and R_{c} are aryl groups, the trisubstituted phosphine dihalide is of a structure as shown in the formula V below: 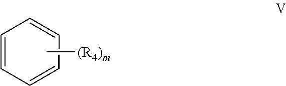
the structural formula as shown in formula V, wherein m is 0, 1, 2, 3, 4, or 5, and R₄ can be hydrogen, a CₙH₂ₙ₊₁ straight-bran or branched-chain alkyl group (1≤n≤10), or halogen.

8. The preparation method of a substituted oxazole compound according to claim 7, characterized in that the said trisubstituted phosphine dihalide is trialkyl phosphine dichloride, triphenyl phosphine dichloride, triphenyl phosphine dibromide, or tris(4-methylphenyl) phosphine dichloride.

9. The preparation method of a substituted oxazole compound according to claim 1, characterized in that:
when the said dehydrating agent is trisubstituted phosphine dihalide, the molar ratio between the trisubstituted phosphine dihalide and the Compound of Formula II is (0.01-5.0):1; preferably, it is (0.1-1.5):1;
when the said dehydrating agent is a combination of trisubstituted phosphine oxide and an acyl halide reagent, the molar ratio between the acyl halide reagent and the Compound of Formula II is (0.1-2.0):1, and that between the trisubstituted phosphine oxide and the Compound of Formula II is (0.01-5.0):1; preferably, the molar ratio between the said acyl halide reagent and the Compound of Formula II is (0.3-1):1, and that between the trisubstituted phosphine oxide and the Compound of Formula II is (0.1-1.5):1;
when the said dehydrating agent is a combination of trisubstituted phosphine dihalide and an acyl halide reagent, the molar ratio between the said acyl halide reagent and the Compound of Formula II is (0.1-2.0):1, and that between the trisubstituted phosphine dihalide and the Compound of Formula II is (0.01-5.0):1; Preferably, the molar ratio between the said acyl halide reagent and the Compound of Formula II is (0.3-1):1, and that between the trisubstituted phosphine dihalide and the Compound of Formula II is (0.1-1.5):1.

10. The preparation method of a substituted oxazole compound according to claim 1, characterized in that the said acyl halide reagent is sulfuryl halide, thionyl halide, oxalyl halide, or carbonyl halide;
the said acyl halide reagent is preferred to be an acyl chloride agent, more preferred to be sulfuryl chloride, thionyl chloride, oxalyl chloride, or carbonyl chloride, and still more preferred to be phosgene, diphosgene, or triphosgene. More preferably, it is phosgene or triphosgene.

11. The preparation method of a substituted oxazole compound according to claim 1, characterized in that when the combination of trisubstituted phosphine oxide and an acyl chloride reagent is used as the dehydrating agent, the substituted oxazole compound can be obtained with the batch synthesis process, and the said acyl halide reagent is dropwise added to the reaction system.

12. The preparation method of a substituted oxazole compound according to claim 1, characterized in that when the combination of trisubstituted phosphine oxide and an acyl chloride reagent is used as the dehydrating agent, the substituted oxazole compound can be obtained with the continuous flow process by feeding the dehydrating agent/organic amine/compound II alone or a mixture of any two thereof continuously.

13. The preparation method of a substituted oxazole compound according to claim 1, characterized in that when the trisubstituted phosphine dihalide compound is used as the dehydrating agent, the substituted oxazole compound can be obtained with the continuous flow process by feeding the dehydrating agent/organic amine/compound II alone or a mixture of any two thereof continuously.

14. The preparation method of a substituted oxazole compound according to claim 12, characterized in that the continuous flow process used in the synthesis can be carried out in a tank continuous reactor, a pipeline continuous reactor, a tower continuous reactor, and/or a microchannel reactor.

15. The preparation method of a substituted oxazole compound according to claim 1, characterized in that the Compound of Formula II is N-ethoxalyl glycine ethyl ester, N-ethoxalyl-α-alanine ethyl ester, N-butoxalyl-α-alanine butyl ester, N-ethoxalyl-α-alanine butyl ester, N-ethoxalyl glycine methyl ester, N-methoxalyl-α-alanine methyl ester, N-ethoxalyl-α-phenylglycine ethyl ester, or N-ethoxalyl-α-alanine methyl ester, or a combination of any two or more thereof.

16. The preparation method of a substituted oxazole compound according to claim 1, characterized in that the cyclization reaction temperature is −20° C.-150° C.; preferably, it is 30-95° C.;
preferably, the cyclization reaction time is 0.2-10 hours.

17. The preparation method of a substituted oxazole compound according to claim 1, characterized in that the Compound of Formula II is cyclized to obtain a reaction liquid; the resulting reaction liquid is further treated to obtain the substituted oxazole compound (I); the said treatment method comprises steps as follows: water is added to the reaction liquid for stratification; an aqueous phase and an organic phase are obtained by extracting the aqueous layer with solvent A and combining organic phases; the resulting organic phase is distilled under atmospheric pressure to recover the solvent A and then under a reduced pressure to obtain the substituted oxazole compound (I); the resulting aqueous phase or the residue of the reduced pressure distillation contains trisubstituted phosphine oxide; the trisubstituted phosphine oxide can be used together with an acyl halide reagent to prepare trisubstituted phosphine dihalide, which functions as a dehydrating agent, or recycled and used as a dehydrating agent directly; the aqueous phase can be neutralized by sodium hydroxide and distilled to recover organic amine.

18. The preparation method of a substituted oxazole compound according to claim 1, characterized in that the said substituted oxazole compound is 4-R₂ substituent-5-R₁ substituent oxy-2-R₃ substituent oxazole; preferably, it is 4-methyl-5-alkoxy-2-R₃ substituent oxazole; more preferably, it is 4-methyl-5-ethyoxyl-2-R₃ substituent oxazole;
preferably, it is 4-methyl-5-ethyoxyl-2-ethoxycarbonyl oxazole, 4-methyl-5-methoxyl-2-methoxycarbonyloxazole, 4-phenyl-5-ethyoxyl-2-ethoxycarbonyl oxazole, or 5-ethyoxyl-2-ethoxycarbonyl oxazole.

19. A preparation method of 4-substituted alkyl-5-substituent oxy oxazole, wherein the said 4-substituted alkyl-5-substituent oxy oxazole is of a structure as shown in formula IV: 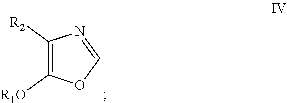
the method comprises steps as follows:
a Compound of Formula II is dissolved in solvent A and cyclized to obtain the substituted oxazole compound (I) in the presence of a dehydrating agent and an organic amine; the substituted oxazole compound (I) is saponified and de-carboxylated to obtain 4-substituted alkyl-5-substituent oxy oxazole (IV);
the dehydrating agent is trisubstituted phosphine dihalide, a combination of trisubstituted phosphine dihalide and an acyl halide reagent, or a combination of trisubstituted phosphine oxide and an acyl halide reagent; 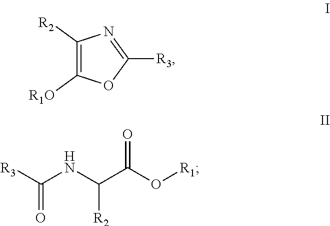
structural formulas I and II of the compounds, wherein:
R₁ can be hydrogen, a CₙH₂ₙ₊₁ straight-chain or branched-chain group (1≤n≤10), aryl, or substituted aryl;
R₂ can be hydrogen, a CₙH₂ₙ₊₁ straight-chain or branched-chain group where (1≤n≤10), aryl, or substituted aryl;
R₃ is —COOR, —CH₂COOR, or —CH₂CH₂COOR, where R is a CₙH₂ₙ₊₁ straight-chain or branched-chain group (1≤n≤10).

20. The preparation method of 4-substituted alkyl-5-substituent oxy oxazole according to claim 19, characterized in that the substituted oxazole compound (I) can be saponified in the presence of alkali to obtain the Compound of Formula III and then decarboxylated under acidic conditions to obtain the Compound of Formula IV; 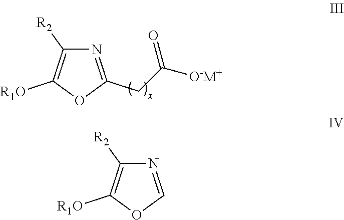
in the structural formula of the Compound of Formula III, the substituents R₁ and R₂ are the same as those in the structural formula of the Compound of Formula II; M is an alkali metal; x is 0, 1, or 2. In the structural formula of the Compound of Formula IV, the substituents R₁ and R₂ are the same as those in the structural formula of the Compound of Formula II;
preferably, the said alkali is the aqueous solution of an alkali metal hydroxide with a mass concentration of 20-30%; the said alkali metal is preferred to be sodium or potassium; the molar ratio between the said alkali and the substituted oxazole compound (I) is 1-1.5:1; the temperature of the said saponification reaction is 20-30° C.;
preferably, the said acidic conditions are created by adjusting the reaction system to a pH of 1-2 through the use of aqueous acid (mass concentration 20-35%); the decarboxylation reaction temperature is 50-70° C.;
preferably, the preparation method of 4-substituted alkyl-5-substituent oxy oxazole, comprising steps as follows:
the Compound of Formula II is cyclized to obtain a reaction liquid; then, water is added to the reaction liquid for stratification; the resulting aqueous layer is extracted with solvent A, and the Compound of Formula I is obtained after combining organic phases and recovering the solvent; alkali is added to the residue for saponification reaction, and stratification is conducted at the end of the reaction; the resulting organic layer is washed with water; the aqueous layers are combined to obtain the resulting solution that contains the Compound of Formula III and decarboxylated by aqueous acid to obtain the Compound of Formula IV; after the Compound of Formula IV is separated, the remaining aqueous phase or organic phase contains trisubstituted phosphine oxide which can be used together with an acyl chloride reagent to prepare trisubstituted phosphine dihalide, which functions as a dehydrating agent, or recycled and used as a dehydrating agent directly.
